(19) United States
(12) Patent Application Publication
(10) Pub. No.: US 2010/0271594 A1
(43) Pub. Date: Oct. 28, 2010

(54) Device and Method for Axial Length Measurement Having Expanded Measuring Function in the Anterior Eye Segment

(75) Inventors: Roland Bergner, Jena (DE); Ingo Koschmieder, Jena (DE); Claus Dreher, Weimar (DE); Rudolf Von Buenau, Jena (DE)

Correspondence Address: FROMMER LAWRENCE & HAUG, 745 FIFTH AVENUE- 10TH FL., NEW YORK, NY, 10151, US

(73) Assignee: Carl Zeiss Meditec AG

(21) Appl. No.: 12/595,592

(22) PCT Filed: Mar. 28, 2008
(86) PCT No.: PCT/EP2008/002457

§ 371 Date: Jul. 7, 2010

(30) Foreign Application Priority Data
Apr. 13, 2007 (DE) 10207017599.1

(51) Int. Cl. A61B 3/117; A61B 3/14

(52) U.S. Cl. 351/206; 351/214; 351/246

(57) ABSTRACT

The present invention is directed to a solution for measuring geometric parameters in the eye which are required for calculating the refractive power of intraocular lenses. The device according to the invention for axial length measurement which acquires axial length, anterior corneal radii, anterior chamber depth, and other parameters in the anterior eye segment includes a control unit, a first measuring device for determining axial length, and an additional measuring device which acquires a plurality of structures in the anterior segment (such as the cornea, anterior chamber, and lens) and which has at least one illumination unit and at least one image recording unit. By determining additional partial-distance parameters of the anterior eye segments, the IOL can be calculated with high precision even after refractive surgery in which the natural relationship between the radii of the anterior and posterior corneal surfaces is extensively altered by corneal surgery.

The present application claims priority from PCT Patent Application No. PCT/EP2008/002457 filed on Mar. 28, 2008, which claims priority from German Patent Application No. DE 10 2007 017 599.1 filed on Apr. 13, 2007, the disclosure of which is incorporated herein by reference in its entirety.

### BACKGROUND OF THE INVENTION

1. Field of the Invention

The present invention is directed to a solution for measuring geometric parameters in the eye which are required for calculating the refractive power of intraocular lenses. These measurements, known under the heading of biometry, are also important particularly for the calculation of intraocular lenses following refractive surgery on the cornea.

2. Description of Related Art

Numerous solutions based on different technical principles are known from the prior art.

PCI in the form of the IOL Master is presently the established benchmark for high-precision axial length measurement. In addition to axial length (AL), the IOL Master supplies three additional measurements—the central radii of the anterior surface of the cornea (K), anterior chamber depth (ACD), and limbus diameter (WTW)—various combinations of which are incorporated in modern IOL calculation formulas. For example, the Haigis formula uses input quantities AL, K and ACD; the Holladay 2 formula also uses the WTW parameter.

In this connection, FIG. 1 shows the basic construction of an IOL Master according to DE 198 57 001 A1. In order to measure axial length AL, the light emitted by the laser diode 2 is imaged onto the patient's eye 1 by a Michelson interferometer 3 and a beamsplitter cube 8. The Michelson interferometer 3 comprises a stationary reference arm with a corner cube 4, an adjustable measuring arm with a displaceable corner cube 5, and a beamsplitter cube 6 for superimposing the two reflected beam components. The path length differences are determined by a path measuring system 7. The partial beams reflected by the cornea and retina of the eye 1 are superimposed and are imaged on an avalanche photodiode 10 by beamsplitter cube 8, achromatic lens 9 and beamsplitter 10. In order to observe the eye 1 and the occurring reflections, a portion of the light coming from the eye 1 is directed to a receiver 12. Other parameters of the eye needed for calculating the required IOL are determined by keratometer measurement (light sources 13 and 15 with illumination optics 14 and 16) and VKT measurement (light source 17 with illumination optics 18).

Various publications have postulated that a more accurate post-operative prediction of IOL positioning would be possible with the help of additional measurements such as, e.g., the thickness of the natural lens and possibly also the lens radii. This in turn would allow a more accurate calculation of the IOL thickness and, therefore, reduced scattering of the post-operative refraction results.

The determination of axial length can also be improved in principle by measuring axial partial distances such as lens thickness and cornea thickness because the refractive indexes of the various media in the eye differ but are usually represented by an individual effective refractive index when converting from optical path length to physical path length. Therefore, by taking partial distances and their different refractive indexes into account it is possible to determine the axial length with greater precision. Further, a number of authors take the approach of ray tracing which incorporates nonparaxial imaging in the optimization of the IOL. This approach (also known as non-formula IOL calculation) requires not only accurate design data for the intraocular lenses used, but also the full description of the optical surfaces in the eye. In particular, the shape of the anterior and posterior corneal surfaces must be known exactly.

Finally, the problem of IOL calculation following refractive corneal surgery (e.g., RK, CK, PRK, and LASIK) is increasingly pervasive. In these cases, the basic problem is that corneal surgery has a lasting effect on the natural relationship between the radii of the anterior and posterior surfaces of the cornea (the so-called Gullstrand ratio) which is implicit in all current IOL calculation formulas. Further, methods such as RK and CK as well as PRK and LASIK procedures with small or decentralized treatment zones result in highly multifocal corneas whose refractive power can be determined only conditionally by measurement at a series of discrete measuring points.

The literature describes a range of calculation formulas for post-refractive eyes. The Haigis-L formula implemented in the IOL Master makes use of the relatively paraxial measurements of the keratometer of the IOL Master and, on the average, delivers outstanding results for eyes after myopic LASIK.

The Oculus Pentacam, which is based on rotating Scheimpflug imaging, provides a complete characterization of the anterior and posterior surfaces of the cornea and measurement of anterior chamber depth and lens thickness. With the aid of the so-called Holladay report, this makes it possible to determine the total refractive power of the cornea, even in post-refractive eyes.

DE 198 12 297 C2 describes a combination device by which the eye length, central corneal radii and anterior chamber depth of the human eye can be measured. The present invention aims beyond this to acquire additional parameters in the anterior eye segment.

DE 299 13 601 U1, DE 299 13 602 U1, and DE 299 13 603 U1 describe a Scheimpflug camera which is used to display the anterior eye segment. While the literature describes the use of a device of this type in connection with the determination of intraocular lenses, eye length measurement is not possible with the device. In contrast, the devices and methods described herein aim to determine all of the necessary parameters with one device.

The system shown in U.S. Pat. No. 6,634,751 uses expressly two separate devices for measuring the parameters in the anterior eye segment and for eye length measurement and calculates an intraocular lens based on these measurements. In contrast, with the present invention both measurements are carried out by the same device.

Similar to the present invention, DE 198 52 331 shows a device which combines interferometric axial length measurement with an additional measuring device for acquiring additional parameters. However, the additional measuring device is always expressly a keratoscope system. This ring system only allows the anterior surface of the cornea to be measured and is unsuitable for acquiring additional structures in the anterior segment such as, e.g., the posterior corneal surface or the natural lens. In contrast, the additional measuring device in the present invention is capable of acquiring a plurality of structures in the anterior segment simultaneously. According to the invention, this is carried out, for example, by means of slit illumination in connection with a Scheimpflug arrangement.

US 2005/0203422 A1 likewise describes a combination device in which all of the parameters aside from those of the anterior corneal surface are acquired by an interferometric method. In contrast to the present invention, this involves a very extensive outlay in apparatus because of the required scanning equipment, resulting in a substantial cost disadvantage. In the present invention, the additional measurement functions are realized as inexpensively as possible, which is accomplished according to the invention in that a plurality of structures in the anterior segment are acquired simultaneously.

### SUMMARY OF THE INVENTION

It is the object of the present invention to acquire more parameters of the anterior eye segment in addition to the quantities which are usually used, i.e., axial length, anterior corneal radii, anterior chamber depth and limbus diameter, for calculating intraocular lenses in a single compact device. In so doing, special emphasis is placed on the acquisition of measurement data for calculating intraocular lens following corneal surgery. In particular, axial and/or axially parallel partial distances (e.g., cornea thickness, lens thickness) and radii (e.g., anterior and posterior corneal radii in different meridional sections and different optical zones, and anterior and posterior lens radii) are to be determined.

The device according to the invention for axial length measurement which acquires axial length, anterior corneal radii, anterior chamber depth and other parameters in the anterior eye segment comprises a control unit, a first measuring device for determining axial length and an additional measuring device which acquires a plurality of structures in the anterior segment such as the cornea, anterior chamber and lens and which has at least one illumination unit and at least one image recording unit. The solution consists in combining a known axial length measuring method with a sectional image projection method for the anterior eye segment in one compact instrument.

In this connection, the axial length measuring method preferably employs an optical coherence interferometer (PCI, OCT) (operating in the time domain or frequency domain). The sectional image method is preferably an optical method utilizing a structured illumination, e.g., slit illumination, and at least one camera. In so doing, the illuminated plane in the eye, the principal plane of the instrument optics, and the camera plane preferably fulfil the Scheimpflug condition. The slit illumination is preferably generated by LEDs and, in one embodiment form, can comprise an arrangement of discrete (collimated) beams.

The device according to the invention for axial length measurement with expanded measuring function in the anterior eye segment is directed to a solution for measuring geometric parameters in the eye which are required for calculating intraocular lenses. The determination of partial distances and parameters of the anterior eye segment allows a more accurate calculation of the eye length acquired by OCT because the refractive indexes of the individual segments of the eye can be used when converting from optical path length to geometric path length. By determining additional partial-distance parameters of the anterior eye segment, the IOL can be calculated with high precision even after refractive surgery in which the natural relationship between the radii of the anterior and posterior corneal surfaces (the so-called Gullstrand ratio) which is implicit in all current IOL calculation formulas is extensively altered by corneal surgery.

### BRIEF DESCRIPTION OF THE DRAWINGS

FIG. 1 shows the basic construction of an IOL Master (prior art);

FIG. 2 shows the combination of a PCI axial length measurement with an axial slit projector and an image recording unit arranged at an inclination;

FIG. 3 shows an azimuthal arrangement of the image recording optics with corresponding slit orientation;

FIG. 4 shows an azimuthal arrangement of a plurality of image recording optics with corresponding slit orientations;

FIG. 5 shows the combination of PCI axial length measurement with a stationary, axial image recording unit and three stationary, inclined slit projectors;

FIG. 6 shows an azimuthal arrangement of a slit projector with axially arranged image recording optics; and

FIG. 7 shows an azimuthal arrangement of a plurality of slit projectors with axially arranged image recording optics.

### DETAILED DESCRIPTION OF EMBODIMENTS

It is to be understood that the figures and descriptions of the present invention have been simplified to illustrate elements that are relevant for a clear understanding of the present invention, while eliminating, for purposes of clarity, many other elements which are conventional in this art. Those of ordinary skill in the art will recognize that other elements are desirable for implementing the present invention. However, because such elements are well known in the art, and because they do not facilitate a better understanding of the present invention, a discussion of such elements is not provided herein.

The present invention will now be described in detail on the basis of exemplary embodiments.

The device according to the invention for eye measurement which acquires axial length, anterior corneal radii, anterior chamber depth and other parameters in the anterior eye segment comprises a control unit, a first measuring device for determining axial length, and an additional measuring device which acquires a plurality of structures in the anterior segment such as the cornea, anterior chamber and lens and which has at least one illumination unit and at least one image recording unit.

The axial length measurement is based on short coherence interferometry and is also suitable for determining axial and/or axially parallel partial distances in the anterior segment, cornea thickness, anterior chamber depth and lens thickness such as central, paracentral, mid-peripheral and/or peripheral thickness, wherein axially parallel partial distances in the anterior segment are acquired by lateral deflection of the measurement beam or lateral displacement of the measuring device.

The measuring device additionally provided for measuring structures in the anterior segment has at least one illumination unit for generating structured illumination and is suitably arranged for determining axial partial distances, axially parallel partial distances, lateral distances, radii of curvature and/or refracting angles. The structured illumination is slit illumination or fringe projection and is generated by means of semiconductor-based conventional light emitting diodes or by means of one or more optoelectronic light modulators, for example, microdisplays. While LEDs (light emitting diodes) or OLEDs (organic light emitting diodes) are used as semiconductor-based light emitting diodes, DMD type (digital micromirror device) microdisplays or LCOS type (liquid crystal on silicon) reflecting microdisplays are used, for example, as optoelectronic light modulators.

However, the structured illumination can also be produced in the form of a series of focal bundles. In this case, the refracting angles of the focal bundles are determined by the control unit from the image acquired by the image recording unit, and the refractive powers of the refracting surfaces are calculated therefrom.

Different meridional sections through the anterior eye segment can be generated by means of the time-variable slit illumination generated by an individual illumination unit and can be recorded by an image recording unit. In so doing, it is particularly advantageous when the image recording unit and/or the illumination unit are/is arranged at an inclination to the axis of the eye and preferably rotate(s) around the axis of the eye.

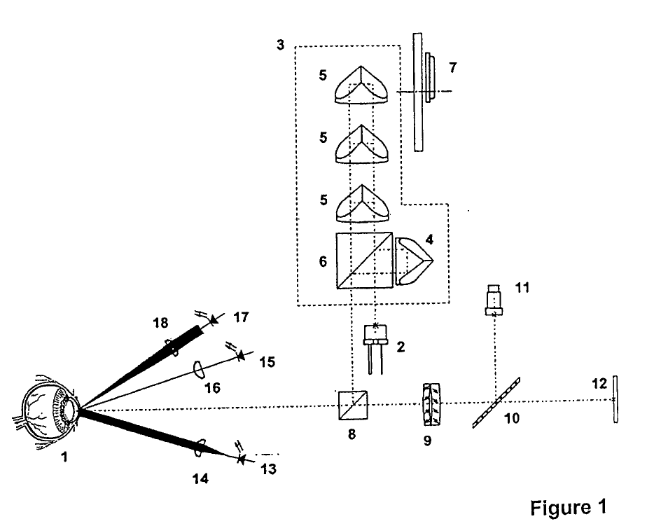

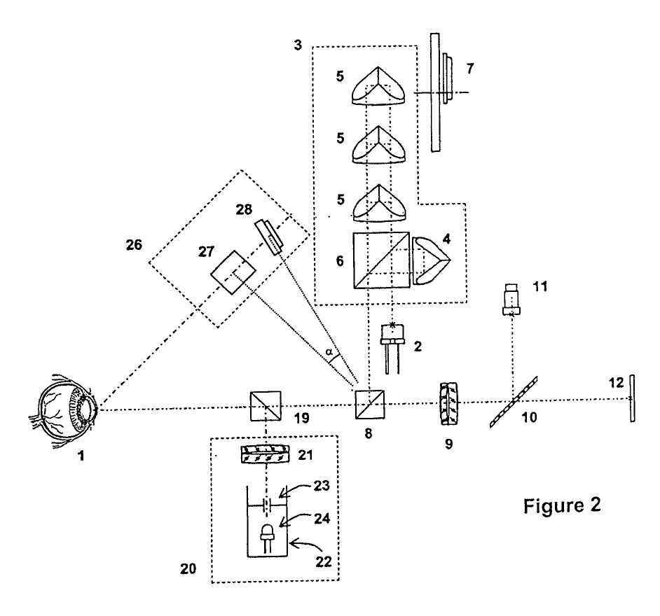

In this regard, FIG. 2 shows the device according to the invention in the form of a combination of a PCI axial length measurement (IOL Master according to FIG. 1) with an axial slit projector 20 and an image recording unit 26 arranged at an inclination.

As is customary, the axial length measurement is carried out centrally along the fixating direction of the patient's eye. In addition, a slit projector 20 is arrange centrally in such a way that the slit image generated by the LED illumination 24 and slit 23 is projected in the eye 1 by the beamsplitter cube 19. The image recording unit 26 comprising imaging optics 27 and camera 28 is positioned at an inclination to the axis of the eye.

In a first embodiment form, the slit lamp 22 comprising the LED illumination 24 and slit 23 is constructed so as to be rotatable so that the generated slit image can rotate around the axis of the eye. In order to record the slit images generated in the eye 1, the image recording unit 26 which is positioned at an inclination to the axis of the eye is constructed in such a way that it rotates around the axis of the eye. The camera 28 of the image recording unit 26 preferably has a sensor chip such as a CCD sensor or CMOS sensor which approximately or exactly fulfils the Scheimpflug condition at the time of the image recording with the illuminated plane in the eye 1. The anterior segment of the eye 1 is scanned in different planes by the slit image generated by the slit projector 20 and is recorded and/or stored at different points in time by the camera 28 for subsequent evaluation.

In a second embodiment form, a plurality of image recording units 26 which are positioned at an inclination to the axis of the eye are positioned in a stationary manner for recording the slit images generated in the eye 1. In this case also, the slit lamp 22 comprising LED illumination 24 and slit 23 is constructed so as to be rotatable so that the generated slit image can rotate around the axis of the eye. In this case too, the cameras 28 of the image recording unit 26 preferably have sensor chips which approximately or exactly fulfil the Scheimpflug condition at the time of the image recording with the illuminated plane in the eye 1. Again, the anterior segment of the eye 1 is scanned in different planes with the slit image generated by the slit projector 20 and is recorded and/or stored by the plurality of cameras 28 for subsequent evaluation.

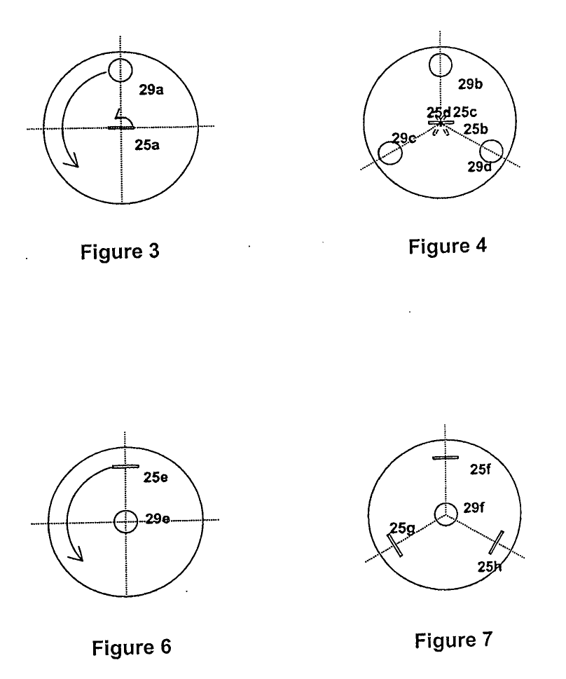

For a clearer illustration and for juxtaposing these two embodiment forms, an azimuthal arrangement of the image recording optics and corresponding slit orientation is shown in FIG. 3 for the first embodiment form and in FIG. 4 for the second embodiment form. Whereas in FIG. 3 both the image 25a of the slit 23 and the imaging beam path (indicated as a circle) 29a of the image recording unit 26 rotate about the axis of the eye and any quantity of slit images may be recorded at different angles, in FIG. 4 the image of the slit 23 rotates but can be recorded by the fixedly positioned image recording units 26 only as images 25b, 25c and 25d. The imaging beam paths of the fixedly positioned image recording units 26 are designated by reference numbers 29b, 29c and 29d.

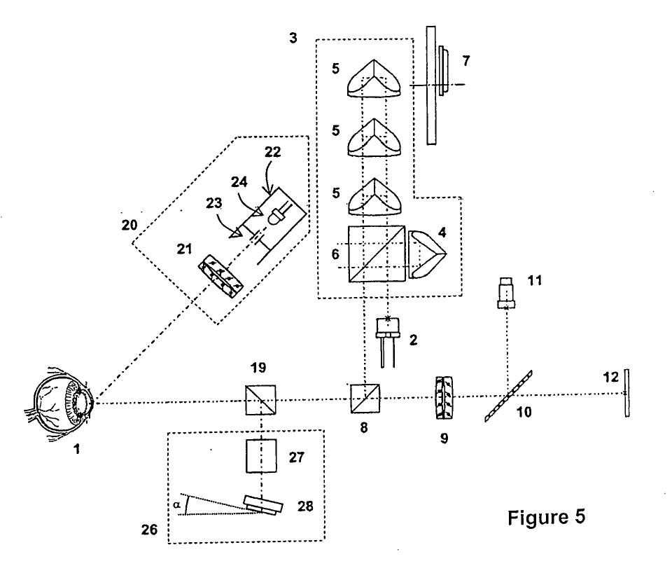

In this connection, FIG. 5 shows the device according to the invention as a combination of PCI axial length measurement with an axial image recording unit and at least one slit projector arranged at an inclination. In this embodiment form, the slit projector 20 can be constructed in such a way that it rotates around the axis of the eye or uses a plurality of slit projectors 20 which are arranged so as to be stationary.

In a third embodiment form of the device according to the invention, an image recording unit 26 is arranged in a stationary manner along this axis and, for example, is coupled in by the beamsplitter cube 19. To generate slit images in the eye 1, at least one slit projector 20 which is positioned at an inclination to the axis of the eye is provided. Accordingly, the slit imaging on the eye 1 is carried out at at least one defined angle to the axis of the eye. In order to image meridional sectional images through the anterior chamber, the patient must gaze at an adjustable fixation point. To record a series of slit images at different azimuthal angles without the slit projector 20 or slit lamp 22 being moved for this purpose, the patient's eye must be sequentially fixated on a quantity of different fixation points. The planes illuminated in the eye 1 by the slit projector 20 at different points in time with the sensor chip of the image recording unit 26 preferably meet the Scheimpflug condition approximately or exactly.

In a fourth embodiment form of the device according to the invention, an image recording unit 26 is arranged in a stationary manner along this axis and, for example, is coupled in by the beamsplitter cube 19. To generate slit images in the eye 1, a plurality of slit projectors 20 are positioned at an inclination to the axis of the eye. The slit imaging on the eye 1 is carried out in this case at a plurality of defined angles relative to the axis of the eye. In this case, the patient need not gaze at an adjustable fixation point for imaging meridional sectional images. The planes illuminated in the eye 1 by the slit projectors 20 at different points in time with the sensor chip of the image recording unit 26 preferably meet the Scheimpflug condition approximately or exactly.

For purposes of a clearer illustration and for juxtaposing these two embodiment forms, an azimuthal arrangement of the image recording optics and corresponding slit orientation is shown in FIG. 6 for the third embodiment form and in FIG. 7 for the fourth embodiment form. Whereas with the stationary imaging beam path 29e of the image recording unit 26 in FIG. 6 the image 25e of the slit 23 rotates so that any quantity of slit images may be recorded at different angles, in FIG. 4 the images 25f, 25g and 25h of the slit 23 and the imaging beam path 29f of the image recording unit 26 indicated by a circle are arranged so as to be stationary so that only three slit images can be recorded.

The control unit, not shown, has means for acquiring and evaluating the angular ratios and distance ratios required for a triangulation at the eye in addition to means for acquiring and evaluating the measured quantities required for axial length measurement. With structured illumination, partial distances, radii and/or refracting angles are determined from the images of the anterior eye segments supplied by the image recording unit.

In addition, it is advantageous when the control unit has means for carrying out dynamic measurement modes such as sequential image recording, differential image recording, or the like, and is able to carry out and evaluate the corresponding measurements simultaneously, wherein the results of one measurement can influence the execution of the other measurement.

In the method for eye measurement according to the invention, the axial length, anterior corneal radii and anterior chamber depth as well as other parameters in the anterior eye segment are acquired, wherein the axial length is determined by means of a control unit and a measuring device, and a plurality of structures in the anterior segment such as the cornea, anterior chamber and lens are acquired by means of a control unit and an additional measuring device which has at least one illumination unit and at least one image recording unit.

The axial length measurement is based on short coherence interferometry and is also suitable for determining axial and/or axially parallel partial distances in the anterior segment, cornea thickness, anterior chamber depth and lens thickness such as central, paracentral, mid-peripheral and/or peripheral thickness, wherein axially parallel partial distances in the anterior segment are acquired by lateral deflection of the measurement beam or lateral displacement of the measuring device. Axial partial distances, axially parallel partial distances, lateral distances, radii of curvature and/or refracting angles are determined by means of the additional measuring device, the latter having at least one illumination unit for generating structured illumination. Structured illumination in the form of a slit projection or fringe projection is generated by the illumination unit by means of semiconductor-based light emitting diodes or by one or more optoelectronic light modulators, for example, microdisplays. While LEDs (light emitting diodes) or OLEDs (organic light emitting diodes), for example, are used as semiconductor-based light emitting diodes, DMD type microdisplays (digital micromirror device) or LCOS (liquid crystal on silicon) type reflecting microdisplays are used, for example, as optoelectronic light modulators.

In the method for eye measurement according to the invention, the structured illumination can also be produced in the form of a series of focal bundles. In this case, the refracting angles of the focal bundles are determined by the control unit from the image acquired by the image recording unit, and the refractive powers of the refracting surfaces are calculated therefrom.

Different meridional sections through the anterior segment of the eye can be generated by the time-variable slit illumination produced by an individual illumination unit and can be recorded by an image recording unit. In so doing, the image recording unit and/or the illumination unit are/is arranged at an inclination to the axis of the eye and can preferably rotate around the axis of the eye. But it is also possible to arrange a plurality of stationary illumination units at an inclination to the axis of the eye and to generate the slit illumination in different meridional sections by changing the fixation direction of the patient's eye.

For this purpose, FIG. 2 shows a possible device for carrying out the method according to the invention, wherein the PCI axial length measurement (IOL Master according to FIG. 1) has been combined with an axial slit projection and an image recorder arranged at an inclination. In so doing, the axial length measurement is carried out centrally along the fixation direction of the patient's eye as is conventional. In addition, slit illumination is carried out centrally to the axis of the eye by means of slit projector 20. The slit image generated by the LED illumination 24 and the slit 23 is projected in the eye 1 by a beamsplitter cube 19. The image recording is carried out by means of an image recording unit 26 which comprises imaging optics 27 and camera 28 and which is arranged at an inclination to the axis of the eye.

In a first embodiment form, the slit lamp 22 comprising the LED illumination 24 and slit 23 is constructed so as to be rotatable so that the generated slit image can rotate around the axis of the eye. In order to record the slit images generated in the eye 1, the image recording unit 26 which is positioned at an inclination to the axis of the eye is constructed in such a way that it rotates around the axis of the eye. The camera 28 of the image recording unit 26 preferably has a sensor chip such as a CCD sensor or CMOS sensor which approximately or exactly fulfils the Scheimpflug condition at the time of the image recording with the illuminated plane in the eye 1. In so doing, the anterior segment of the eye 1 is scanned in different planes with the slit image generated by the slit projector 20 and is recorded and/or stored at different points in time by the camera 28 for subsequent evaluation.

In a second embodiment form, a plurality of image recording units 26 are positioned in a stationary manner at an inclination to the axis of the eye for recording the slit images generated in the eye 1. In this case also, the slit lamp 22 comprising LED illumination 24 and slit 23 is constructed so as to be rotatable so that the generated slit image can rotate around the axis of the eye. In this case too, the cameras 28 of the image recording unit 26 preferably have sensor chips which approximately or precisely meet the Scheimpflug condition at the time of the image recording with the illuminated plane in the eye 1. Again, the anterior segment of the eye 1 is scanned in different planes with the slit image generated by the slit projector 20 and is recorded and/or stored by the plurality of cameras 28 for subsequent evaluation.

For a clearer illustration and for juxtaposing these two embodiment forms, an azimuthal arrangement of the image recording optics and corresponding slit orientation is shown in FIG. 3 for the first embodiment form and in FIG. 4 for the second embodiment form. Whereas in FIG. 3 both the image 25a of the slit 23 and the imaging beam path (indicated as a circle) 29a of the image recording unit 26 rotate about the axis of the eye and any quantity of slit images may be recorded at different angles, in FIG. 4 the image of the slit 23 rotates but can be recorded by the fixedly positioned image recording units 26 only as images 25b, 25c and 25d. The imaging beam paths of the fixedly positioned image recording units 26 are designated by reference numbers 29b, 29c and 29d.

In this connection, FIG. 5 shows another device for carrying out the method according to the invention as a combination of PCI axial length measurement with axial image recording and at least one slit projector arranged at an inclination. Either a slit projector 20 rotating around the axis of the eye or a plurality of slit projectors 20 arranged so as to be stationary are used for this purpose.

In a third embodiment form of the method according to the invention, the image recording unit 26 provided for image recording is arranged along this axis and, for example, is coupled in by the beamsplitter cube 19. To generate slit images in the eye 1, at least one slit projector 20 which is positioned at an inclination to the axis of the eye is provided. Accordingly, the slit imaging on the eye 1 is carried out at least one defined angle relative to the axis of the eye. In order to image meridional sectional images through the anterior chamber, the patient must gaze at an adjustable fixation point. To record a series of slit images at different azimuthal angles without the slit projector 20 or slit lamp 22 being moved for this purpose, the patient's eye must be sequentially fixated on a quantity of different fixation points. The planes illuminated in the eye 1 by the slit projector 20 at different points in time with the sensor chip of the image recording unit 26 preferably meet the Scheimpflug condition approximately or exactly.

In a fourth embodiment form of the method according to the invention, the image recording unit 26 provided for image recording is arranged in a stationary manner along this axis and, for example, is coupled in by the beamsplitter cube 19. To generate slit images in the eye 1, a plurality of slit projectors 20 are positioned at an inclination to the axis of the eye. Accordingly, the slit imaging on the eye 1 is carried out at a plurality of defined angles relative to the axis of the eye. The planes illuminated in the eye 1 by the slit projectors 20 at different points in time with the sensor chip of the image recording unit 26 preferably meet the Scheimpflug condition approximately or exactly.

For purposes of a clearer illustration and for juxtaposing these two embodiment forms, an azimuthal arrangement of the image recording optics and corresponding slit orientation is shown in FIG. 6 for the third embodiment form and in FIG. 7 for the fourth embodiment form. Whereas with the stationary imaging beam path 29e of the image recording unit 26 in FIG. 6 the image 25e of the slit 23 rotates so that any quantity of slit images may be recorded at different angles, in FIG. 4 the images 25f, 25g and 25h of the slit 23 and the imaging beam path 29f of the image recording unit 25 indicated by a circle are arranged so as to be stationary so that only three slit images can be recorded.

The control unit, not shown, has means for acquiring and evaluating the angular ratios and distance ratios required for a triangulation at the eye in addition to means for acquiring and evaluating the measured quantities required for axial length measurement. With structured illumination, partial distances, radii and/or refracting angles are determined from the images of the anterior eye segments supplied by the image recording unit.

In addition, it is advantageous when the control unit has means for carrying out dynamic measurement modes such as sequential image recording, differential image recording, or the like, and is able to carry out and evaluate the corresponding measurements simultaneously, wherein the results of one measurement can influence the execution of the other measurement.

When all of the acquired parameters are evaluated, the required spherical refractive power of an intraocular lens is calculated by the control unit by means of lens calculation formulas and empirical constants or by means of a ray tracing method. In addition to spherical power, a suitable asphericity value is also calculated for an intraocular lens in addition to the required cylindrical refractive power.

The device and method according to the invention for eye measurement provide a solution by which more parameters of the anterior eye segment besides the quantities which are usually used, i.e., axial length, anterior corneal radii, anterior chamber depth and limbus diameter, can be acquired for calculating intraocular lenses, particularly after corneal surgery, in a single compact device. In a particularly advantageous manner, axial and/or axially parallel partial distances (e.g., cornea thickness, lens thickness) and radii (e.g., anterior and posterior corneal radii in different meridional sections and different optical zones, and anterior and posterior lens radii) are determined. In this way, all of the parameters required for calculating and selecting an IOL can be determined with the required precision and with the lowest possible expenditure on apparatus.

Knowledge of the partial distances makes it possible to calculate the eye length acquired by means of PCI with greater precision because the refractive indexes of the individual eye segments can be used when converting from optical path length to geometric path length.

While this invention has been described in conjunction with the specific embodiments outlined above, it is evident that many alternatives, modifications, and variations will be apparent to those skilled in the art. Accordingly, the preferred embodiments of the invention as set forth above are intended to be illustrative, not limiting. Various changes may be made without departing from the spirit and scope of the inventions as defined in the following claims.

### REFERENCE NUMBERS

1 eye
2 laser diode
3 Michelson interferometer, comprising:
4 corner cube
5 corner cube (different positions)
6 beamsplitter cube
7 path measuring system
8 beamsplitter cube
9 achromatic lens
10 beamsplitter
11 receiver
12 camera
13, 15 light source for keratometer
14, 16 illumination optics for keratometer
17 light source for VKT measurement
18 illumination optics for VKT measurement
19 beamsplitter cube
20 slit projector, comprising:
21 projection optics
22 rotatable slit lamp, comprising:
23 slit
24 LED illumination
25 images of the slit 23
26 image recording unit, comprising:
27 imaging optics
28 camera
29 imaging beam paths of the image recording unit 26

1. An apparatus for eye measurement which acquires axial length, anterior corneal radii, anterior chamber depth and other parameters in an anterior eye segment comprising:
a control unit;
a first measuring device which determines axial length; and
an second measuring device which acquires a plurality of structures in the anterior segment, and which has at least one illumination unit and at least one image recording unit.

2. The apparatus according to claim 1;
wherein the first measuring device is based on short coherence interferometry.

3. The apparatus according to claim 2;
wherein the first measuring device also determines axial distances and/or axially parallel partial distances in the anterior segment, cornea thickness, anterior chamber depth, and lens thickness.

4. The apparatus according to claim 3;
wherein axially parallel partial distances in the anterior segment are acquired by lateral deflection of the measurement beam or lateral displacement of the first measuring device.

5. The apparatus according to claim 1;
wherein the second measuring device determines axial partial distances, axially parallel partial distances, lateral distances, radii of curvature, and/or refracting angles, and has at least one illumination unit for generating structured illumination.

6. The apparatus according to claim 5;
wherein the structured illumination is slit illumination or fringe projection, and is generated by conventional light emitting diodes (“LEDs”) or light diodes based on organic materials (“OLEDs”).

7. The apparatus according to claim 5;
wherein the structured illumination is slit illumination or fringe projection, and is generated by one or more spatial light modulators.

8. The apparatus according to claim 5;
wherein the structured illumination is in the form of a series of focal bundles; and
wherein refracting angles of the focal bundles are determined by the control unit based on the image acquired by the image recording unit, and refractive powers of refracting surfaces are calculated based on the refracting angles.

9. The apparatus according to claim 5;
wherein an individual illumination unit generates time-variable slit illumination in different meridional sections through the anterior segment.

10. The apparatus according to claim 5;
wherein a plurality of illumination units are arranged at an inclination to the axis of the eye and generate slit illumination in different meridional sections by changing the fixating direction of the eye.

11. The apparatus according to claim 1;
wherein at least one image recording unit contains a sensor chip which approximately or exactly fulfils the Scheimpflug condition at the time of the image recording with the illuminated plane in the eye.

12. The apparatus according to claim 11;
wherein the image recording unit and/or the illumination unit are/is arranged at an inclination to the axis of the eye.

13. The apparatus according to claim 11;
wherein a plurality of stationary image recording units are provided which approximately or precisely meet the Scheimpflug condition at different times with the illuminated plane in the eye.

14. The apparatus according to claim 11;
wherein a plurality of stationary illumination units are provided whose illuminated planes in the eye approximately or precisely meet the Scheimpflug condition at different times with the sensor chip.

15. The apparatus according to claim 11;
wherein exactly one illumination unit is provided whose structured illumination scans the anterior segment in different planes and in so doing meets the Scheimpflug condition at different times with the sensor chip.

16. The apparatus according to claim 1;
wherein an adjustable fixating unit for the patient is provided for deflecting the gaze.

17. The apparatus according to claim 16;
wherein an accommodation state of the eye can be influenced additionally by means of the fixating unit.

18. The apparatus according to claim 1, further comprising:
means for acquiring and evaluating the angular ratios and distance ratios required for a triangulation at the eye.

19. A method for eye measurement by which an axial length, anterior corneal radii, anterior chamber depth, and other parameters in an anterior eye segment are acquired, the method comprising:
determining the axial length by means of a control unit and a first measuring device; and
acquiring a plurality of structures in the anterior segment by means of the control device and an second measuring device which has at least one illumination unit and at least one image recording unit.

20. The method according to claim 18;
wherein the measurement of axial length is carried out by means of short coherence interferometry.

21. The method according to claim 20;
wherein axial distances and/or axially parallel partial distances in (1) the anterior segment, (2) the central, zonal, and/or peripheral cornea thickness, (3) the anterior chamber depth, and (4) the lens thickness are determined by means of short coherence interferometry.

22. The method according to claim 21;
wherein axially parallel partial distances in the anterior segment are acquired by lateral deflection of the measurement beam or lateral displacement of the first measuring device.

23. The method according to claim 19;
wherein axial partial distances, axially parallel partial distances, lateral distances, radii of curvature, and/or refracting angles are determined by the second measuring device; and
wherein the second measuring device has at least one illumination unit for generating structured illumination.

24. The method according to claim 23;
wherein the structured illumination is slit illumination or fringe projection and is generated by conventional light emitting diodes (LEDs) or light diodes based on organic materials (OLEDs).

25. The method according to claim 23;
wherein the structured illumination is slit illumination or fringe projection and is generated by one or more spatial light modulators.

26. The method according to claim 23;
wherein the structured illumination is in the form of a series of focal bundles; and
wherein the refracting angles of the focal bundles are determined by the control unit based on the image acquired by the image recording unit, and the refractive powers of the refracting surfaces are calculated based on the refracting angles.

27. The method according to claim 23;
wherein an individual illumination unit generates time-variable slit illumination in different meridional sections through the anterior segment.

28. The method according to claim 23;
wherein a plurality of illumination units are arranged at an inclination to the axis of the eye and generate slit illumination in different meridional sections by changing the fixating direction of the patient's eye.

29. The method according to claim 23;
wherein at least one image recording unit contains a sensor chip which approximately or exactly fulfils the Scheimpflug condition at the time of the image recording with the illuminated plane in the eye.

30. The method according to claim 29;
wherein the image recording unit and/or the illumination unit are/is arranged at an inclination to the axis of the eye.

31. The method according to claim 29;
wherein a plurality of stationary image recording units are provided which approximately or precisely meet the Scheimpflug condition at different times with the illuminated plane in the eye.

32. The method according to claim 29;
wherein a plurality of stationary illumination units are provided whose illuminated planes in the eye approximately or precisely meet the Scheimpflug condition at different times with the sensor chip.

33. The method according to claim 29;
wherein exactly one illumination unit is provided whose structured illumination scans the anterior segment in different planes and in so doing meets the Scheimpflug condition at different times with the sensor chip.

34. The method according to claim 18;
wherein the gaze of the patient is deflected by means of an adjustable fixating unit.

35. The method according to claim 34;
wherein the accommodation state of the eye can be influenced additionally by means of the fixating unit.

36. The method according to claim 18;
wherein the angular ratios and distance ratios required for a triangulation at the eye are acquired and evaluated.

37. The method according to claim 18;
wherein a required spherical refractive power of an intraocular lens is calculated based on the acquired data by lens calculation formulas and empirical constants or by means of a ray tracing method.

38. The method according to claim 37;
wherein a required cylindrical refractive power of an intraocular lens is calculated based on the acquired data in addition to the spherical refractive power.

39. The method according to claim 37;
wherein a suitable asphericity value is calculated for an intraocular lens based on the acquired data in addition to determining the required spherical refractive power.
